(19) United States
(12) Patent Application Publication
(10) Pub. No.: US 2011/0213042 A1
(43) Pub. Date: Sep. 1, 2011

(54) METHOD FOR THE PREPARATION OF A HYDROCARBON SYNTHESIS CATALYST AND THE USE THEREOF IN A HYDROCARBON SYNTHESIS PROCESS

(76) Inventor: Johan Labuschagne, Free State (ZA)

(21) Appl. No.: 12/671,657

(22) PCT Filed: Jul. 28, 2008
(86) PCT No.: PCT/IB08/53019

§ 371 Date: Apr. 28, 2010

(30) Foreign Application Priority Data
Aug. 2, 2007 (ZA) 2007/06449

(51) Int. Cl. B01J 23/78; B01J 23/02; C07C 1/02

(52) U.S. Cl. 518/728; 502/340; 502/344; 502/328; 502/330

(57) ABSTRACT

This invention relates to a method for the preparation of a hydrocarbon synthesis catalyst, preferably, a Fischer Tropsch synthesis catalyst. The invention also extends to the use of a catalyst prepared by the method according to the invention in a hydrocarbon synthesis process, preferably, a Fischer Tropsch synthesis process. According to a first aspect of the invention there is provided a method for the preparation of a hydrocarbon synthesis catalyst, the method including the steps of: (a) providing a melt including a mixture of at least one metal iron oxide and a catalyst promoter selected from the group consisting of at least one of a source of an alkali metal and a source of an alkali earth metal; (b) subjecting the melt to a fluid stream so as to thereby disperse the melt into droplets including the metal iron oxide and the catalyst promoter; and (c) quenching the droplets of the melt so as to form the hydrocarbon synthesis catalyst in the form of solid particles including the metal oxide and the catalyst promoter.

### FIELD OF THE INVENTION

This invention relates to a method for the preparation of a hydrocarbon synthesis catalyst, preferably, a Fischer Tropsch synthesis catalyst. The invention also extends to the use of a catalyst prepared by the method according to the invention in a hydrocarbon synthesis process, preferably, a Fischer Tropsch synthesis process.

### BACKGROUND TO THE INVENTION

A Fischer-Tropsch process comprises the hydrogenation of CO in the presence of a catalyst based on metals, such as iron, cobalt and ruthenium. The products formed from this reaction are water, gaseous, liquid and waxy hydrocarbons which may be saturated or unsaturated. Oxygenates of the hydrocarbons such as alcohols, acids, ketones and aldehydes are also formed. The carbon number distribution of the products follow the well-known Anderson-Schulz-Flory distribution.

The Fischer-Tropsch process can be described as a heterogeneous surface catalyzed polymerization reaction. The reaction entails the hydrogenation of carbon monoxide over certain metal catalysts to form a range of hydrocarbons as represented by the following general equation:

CO+H₂→(CH₂)ₙ+H₂O

A heterogeneous Fisher-Tropsch process may be conveniently categorized as either a high temperature Fischer-Tropsch (HTFT) process or a low temperature Fischer-Tropsch (LTFT) process. The HTFT process can be described as a two-phase Fischer-Tropsch process. It is usually carried out at a temperature from 250° C. to 400° C. and the catalyst employed is usually a fused iron-based catalyst.

The metals used as catalysts for the Fischer-Tropsch synthesis are generally promoted with group IA and HA non-ferrous elements in order to enhance the activity and selectivity of the catalyst. There are two main groups of promoters, namely, structural and chemical promoters. Structural promoters increase and stabilize the available active metal surface area, that is they give structural stability and porosity to the catalyst matrix. Chemical promoters generally affect the product selectivity.

The iron-based catalyst for the high temperature Fischer-Tropsch (HTFT) process is usually prepared by a fusion process. This entails melting iron oxides with chemical and structural promoters in an electric arc furnace. The chemistry involved in the fusion process is complicated and difficult to control.

Under the high temperatures required for fusion of the iron oxides, some of the structural promoters and impurities in the raw materials, particularly silica, combine with a significant portion of the chemical promoters in solid-state reactions. Thus the fused catalyst is relatively unresponsive to chemical promotion and optimization of the selectivity of this catalyst is restricted. The high fusion temperature also results in the volatilization of some of the promoters, such as potassium oxide.

Once the promoters have been fused with the iron, the melt is subjected to a solidification process and segregation of the promoters takes places. This results in the establishment of a promoter concentration gradient from the segment of material solidifying first to that segment solidifying last which consequently gives rise to undesirably higher levels of promoter in the latter section. As a result of this, the promoters are not homogeneously distributed and the effective catalyst alkalinity varies with particle size in the milled catalyst. Smaller particles have a much higher catalyst alkalinity compared to larger particles. It is believed that this high catalyst alkalinity in small particles and high concentration of promoters along the grain boundaries in larger particles are the main reasons for not only high carbon make but also high acid selectivities during synthesis. This high carbon make results in a fast decline in catalyst bed density in the fluidised reactors and hence places a limit on the catalyst lifetime.

Over the last 20 years, both Rapid Solidification Processing (RSP) and Spray Deposition Processing (SDP) have received considerable attention as alternative routes for the production of highly reactive alloys and high performance materials. RSP involves the quenching of molten metal to a solid state at a very high rate via droplet formation. The method of liquid metal droplet solidification is utilized for the production of a broad spectrum of ferrous and non-ferrous metal powders on a commercial scale as this method allows the characteristics of such powder, in particular size, size distribution and shape, to be controlled.

The primary use of consolidated metal powders is in fabricating net-shape products. To produce components with superior mechanical and corrosion properties, there is greater demand for high quality powder; powder that is clean, that has desired size distribution and morphology, and that is produced in a cost-effective manner. Besides the traditional applications of metal powders employed within the automotive, medical and defense industries, metal powders are seeing increased use in other areas. They are used as feedstock for thermal spray coatings, solid freeform fabrication and rapid prototyping processes and in electronic and magnetic applications. Fine-pitch interconnections, high temperature solders and some magnetic alloys use metal powders. Metal powders are an essential component of, inter alia, magnetic recording tapes, electrical conducting tapes, capacitative tapes and electro-magnetic interference shielding. For most of the electronic and magnetic applications, powders in the very fine size range (<10 μM) are desirable.

The inventors have found that surprisingly Rapid Solidification Processing is a useful alternative method for the preparation of a Fischer Tropsch catalyst wherein, inter alia, the disadvantages set out above with reference to the distribution of promoters in the catalyst are avoided or at least minimised. This and other advantages are discussed more fully below.

### DISCLOSURE OF THE INVENTION

According to a first aspect of the invention there is provided a method for the preparation of a hydrocarbon synthesis catalyst, the method including the steps of:
(a) providing a melt including a mixture of at least one metal iron oxide and a catalyst promoter selected from the group consisting of at least one of a source of an alkali metal and a source of an alkali earth metal;
(b) subjecting the melt to a fluid stream so as to thereby disperse the melt into droplets including the metal iron oxide and the catalyst promoter; and
(c) quenching the droplets of the melt so as to form the hydrocarbon synthesis catalyst in the form of solid particles including the metal oxide and the catalyst promoter.

The metal oxide is preferably iron oxide and may be in the form of magnetite (Fe₃O₄). It will be appreciated that reference to iron oxide extends to any oxide of iron.

The melt may include more than one iron oxide and may include a mixture of iron oxides. In an embodiment of the invention the mixture of iron oxides may comprise a mixture of magnetite and wüstite (FeO). In an alternate embodiment of the invention, the mixture of iron oxides may comprise a mixture of magnetite and hematite (Fe₂O₃).

In an embodiment of the invention, the melt includes 60% to 100% (wt %) of magnetite, preferably 60% to 80%; and 0% to 40% (wt %), preferably 20% to 40% of wüstite. Accordingly, the catalyst contains between 68% to 73% (wt %) of total iron metal (Fe).

In one embodiment of the invention, the iron oxide may be mixed with non ferrous metal components. The non ferrous metal components may be selected from a source of a group IIIA or IVA element. The components may be present in the amount of 0% to 1.0% (wt %).

In an embodiment of the invention, the source of the alkali metal may be selected from a source of elements from Group IA. In a preferred form of the invention, the source of alkali metal may be selected from at least one of the group consisting of sodium carbonate and potassium carbonate.

The source of the alkali earth metal may be selected from a source of elements from Group IIA. In a preferred form of the invention, the source of alkali earth metal may be selected from at least one of the group consisting of magnesium carbonate and calcium carbonate.

In an embodiment of the invention, the catalyst promoter may comprise a mixture of a source of alkali metals and a source of alkali earth metals. In a preferred form of the invention, the catalyst promoter comprises magnesium carbonate, calcium carbonate, sodium carbonate and potassium carbonate. The hydrocarbon synthesis catalyst comprises between 0.01% to 4.0% (wt % of the total hydrocarbon synthesis catalyst composition).

The melt may also include trace impurities that stem from the source of iron used, for example mill scale. Such impurities may be any one or more of the following: SiO₂, Al₂O₃, MnO₂, Cr₂O₃, TiO₂ or V₂O₅. The trace impurities may be present in the amount of 5.0 wt %, preferably below 2.5 wt % and more preferably below 1.0 wt % of the total composition of the catalyst.

The melt may be subjected to a fluid stream that may be a gas, preferably nitrogen or a liquid, preferably water. The fluid stream may be pressurised.

In a preferred form of the invention, pressurised water at a pressure of 50 to 150 bar, preferably 75 bar is used to disperse the melt into droplets.

In an embodiment of the invention, an atomizer is used to disperse the melt into droplets.

The droplets of the melt are cooled from a temperature of 1600° C. to 1700° C., preferably at 1650° C., to a temperature of 15° C. to 20° C., so as to form the hydrocarbon synthesis catalyst in the form of solid particles. The cooling takes places rapidly, typically between 1 to 2 seconds. The cooling step herein described is often referred to as quench cooling, wherein a molten metal stream is disintegrated into droplets which are then very rapidly cooled into solid particles, i.e. cooling rates of 10⁵-10⁶ K/s can be obtained. It will be appreciated that quench cooling via water atomization is but one technique that may be used for purposes of rapid solidification.

The cooling of the droplets takes place as a result of the fact that the solid particles have a small mass and high heat transfer rate.

In an embodiment of the invention, and where the fluid stream is a liquid, preferably under pressure, the solid particles formed may be separated from the liquid by either one or a combination of the following techniques, namely magnetic separation, vacuum filtration, drying or any other conventional means. In a preferred form of the invention the solid particles are air dried in a rotary oven.

The solid particles may be substantially spherical in shape and may have a particle size range of 0.5 to 500 micron, preferably 5 to 250 micron and most preferably between 10 to 150 micron. The BET surface area of the solid particles may be smaller than 5 m²/g. It is envisaged that the surface area will not be smaller than 1 m²/g.

The catalyst promoter may be homogenously distributed within the solid particles and it is envisaged that each particle, irrespective of the size thereof, shall have a homogeneous distribution of catalyst promoter therein.

The inventors believe that by following the process according to the invention, segregation of the catalyst promoters is inhibited such that the homogeneous spread of catalyst promoter in the individual solid particles of hydrocarbon synthesis catalyst will provide substantially the same hydrocarbon product selectivity and will allow for better quality control in respect of the composition of the hydrocarbon synthesis catalyst.

It is also believed that the hydrocarbon synthesis catalyst prepared according to the method herein described has at least similar, if not better, mechanical strength when compared to conventional hydrocarbon synthesis catalysts prepared by means of milling a typically fused iron oxide catalyst, which is prepared in the conventional manner known in the art. The inventors envisage that as a result, subsequent catalyst break-up in the synthesis reactor and catalyst carry over with hydrocarbon products will be minimised.

It will be appreciated that the above method also provides the advantage that the hydrocarbon synthesis catalyst is formed directly from the melt and may be dispersed into solid particles of a desired particle size distribution, by varying the pressure of the fluid stream, so that the steps in conventional methods for preparing fused hydrocarbon synthesis catalysts, such as casting, crushing, milling, classification and cyclone separation are done away with thereby decreasing the production and maintenance costs of the overall catalyst manufacturing process.

In an embodiment of the invention, the hydrocarbon synthesis catalyst is a Fischer Tropsch catalyst. Preferably, it is a High Temperature Fischer Tropsch catalyst.

The hydrocarbon synthesis catalyst may be activated by means of reduction. In an embodiment of the invention the solid particles may be subjected to a heat treatment step so as to reduce the metal oxide to a metal having an oxidation state of zero. Preferably the heat treatment step reduces the metal oxide, being iron oxide in a preferred embodiment of the invention, to iron with an oxidation state of zero so as form a reduced hydrocarbon synthesis catalyst.

The heat treatment step may be carried out in the presence of a reducing gas. In an embodiment of the invention, the reducing gas is at a pressure of 15 to 25 bar. The reducing gas may be hydrogen and/or carbon monoxide.

In an embodiment of the invention, the heat treatment step may be carried out at a temperature of 350° C. to 450° C., preferably 450° C. The heat treatment step may be carried out for 12 to 24 hours, preferably 12 hours.

The BET surface area of the reduced hydrocarbon synthesis catalyst may be from 20 to 30 m²/g and the particles will still have a substantially spherical shape.

It is believed by the inventors that the substantially spherical shape of the solid particles of hydrocarbon synthesis catalyst shall improve the flow properties of the catalyst when used in a hydrocarbon synthesis process, preferably in a Fischer Tropsch Process, and more preferably in a fluidised High Temperature Fischer Tropsch Process. This in turn will result in better fluidization in the reactor zone and stable operation of the cyclones in the commercial SAS reactors due to a lower change in pressure in the dipleg of the cyclone.

The reduced hydrocarbon synthesis catalyst may be subjected to a conditioning step. The conditioning step may be carried out by the stepwise replacement of the reducing gas with synthesis gas. Preferably, the reducing gas, in the form of hydrogen, is replaced in a stepwise fashion with carbon monoxide.

In an embodiment of the invention, the reducing gas is replaced with carbon monoxide at a pressure of from 15 bar to 25 bar. The conditioning step may take place at a temperature of from 250° C. to 350° C. and may be carried out for a period of 24 hours.

The conditioning step may be carried out when the reducing gas is hydrogen and its stepwise replacement is with carbon monoxide until the H₂:CO molar ratio in the total synthesis gas feed is in the range of 5:1 to 1:5, preferably 4:1.

According to a second aspect of the invention, there is provided a hydrocarbon synthesis catalyst prepared according to the process set out herein.

According to a third aspect of the invention, there is provided the use of a hydrocarbon synthesis catalyst in a Fischer Tropsch reaction. Preferably the FT reaction is an HTFT reaction and preferably the hydrocarbon synthesis catalyst is reduced.

According to a fourth and further aspect of the invention, there is provided a two phase High Temperature Fischer Tropsch process for the conversion of a feed of H₂ and at least one carbon oxide to hydrocarbons containing at least 40% on a mass basis of hydrocarbons with five or more carbon atoms; the conversion being carried out by contacting the H₂ and the at least one carbon oxide in the presence of a hydrocarbon synthesis catalyst prepared by a method comprising the steps of:
(a) providing a melt including a mixture of at least one metal oxide and a catalyst promoter selected from the group consisting of at least one of a source of an alkali metal and a source of an alkali earth metal;
(b) subjecting the melt to a fluid stream so as to thereby disperse the melt into droplets including the metal oxide and the catalyst promoter;
(c) quenching the droplets of the melt so as to form the hydrocarbon synthesis catalyst in the form of solid particles including the metal oxide and the catalyst promoter; and
(d) subjecting the solid particles of the hydrocarbon synthesis catalyst of step (c) to a heat treatment step so as reduce the metal oxide to a metal having an oxidation state of zero.

The synthesised hydrocarbons contain, on a mass basis, at least 40%, more preferably at least 50% and most preferably at least 60% C₅₊ hydrocarbons.

The temperature range for the HTFT hydrocarbon synthesis process may be between 280° C. to 400° C., preferably above 300° C., typically from 300° C. to 370° C., and even from 330° C. to 350° C. The pressure may be from 10 to 60 bar, typically 15 to 30 bar, and usually at about 20 to 25 bar.

The reaction may be carried out in any suitable reactor, preferably a fluidised bed reactor, more preferably in a fixed fluidised bed reactor.

The composition of the total synthesis gas feed comprises an H₂:CO ratio of 5:1 to 1:5, preferably 4:1. Typically, the feed of synthesis gas may also comprise about 1% to 25% volume percent CO₂; N₂ and/or methane.

### BRIEF DESCRIPTION OF THE DRAWINGS

FIG. 1: Secondary Scanning Electron Microscopy (SEM) Image showing the morphology of catalyst particles produced according to the invention at 75 bar water pressure.

FIG. 2: Secondary SEM Image showing the morphology of conventional fused, cast and milled catalyst particles.

FIG. 3a: Back Scatter SEM image showing the morphology of a polished cross section of a catalyst particle prepared according to the invention.

FIG. 3b: Energy Dispersive X-Ray (EDX) line scan showing the distribution of promoters along the cross section A-A of the catalyst particle, of FIG. 3a, prepared according to the invention.

FIG. 4: EDX line scan showing the distribution of promoters of a catalyst particle, of FIG. 5c, of conventional fused, cast and milled catalyst particles.

FIG. 5a: Backscatter SEM image of a polished section through conventional cast catalyst particles.

FIG. 5b: Backscatter SEM image of a polished section through conventional cast catalyst particles.

FIG. 5c: Backscatter SEM image of a polished section through conventional cast catalyst particles.

FIG. 6: Results of impact attrition tests for the hydrocarbon synthesis catalyst prepared according to the invention and a standard fused catalyst known in the art.

This invention will now be further described by means of the following non-limiting examples.

### EXAMPLES

Unless otherwise specified, the data and graphs discussed hereunder are in respect of the hydrocarbon synthesis catalyst (atomized catalyst) and the standard fused catalyst prepared below.

Secondary Backscatter SEM images and EDX linescans were obtained using the following SEM parameters:

| | |
|---|---|
| Operating voltage | 20-25 kV |
| Working distance | 13-17 mm |
| Magnification | 150X-2000X |

### Example 1

### Preparation of a Standard Conventional Fused Hydrocarbon Synthesis Catalyst (Hereinafter Referred to as “Standard Fused Catalyst”) and a Hydrocarbon Synthesis Catalyst According to the Invention

A standard fused catalyst was prepared by fusing iron oxide in the form of magnetite together with the chemical promoter K₂O and the structural promoters MgO or Al₂O₃ in an electric arc furnace at a temperature of approximately 1650° C. The melt was then poured into pans on a continuous casting belt with the result that segregation of the promoter concentration occurs in the cast material during solidification such that the concentration gradient varies from that segment of the material solidifying first to that segment of the material solidifying last. The resultant ingots of fused catalyst were then crushed into pieces and milled. It will be appreciated that the standard fused catalyst was prepared in a manner well known in the art.

A hydrocarbon synthesis catalyst according to the invention was prepared by providing a melt comprising hematite, magnetite and wüstite, the melt further including the catalyst promoters of calcium carbonate, magnesium carbonate, sodium carbonate and potassium carbonate. Non ferrous metals of silicon and aluminum were present as a result of refractory material present in the mill scale. All of these were mixed together and fused at a temperature of 1650° C. in an AC electric arc furnace having a freeze lining in order to prevent contamination of the melt with refractory materials from the wall of the furnace.

The melt as described above was then fed into an atomizer where the melt was subjected to jets of pressurised water in order to disperse the melt into droplets. The pressure of the water was varied from 50 bar to 150 bar for reasons more fully discussed below.

The resultant droplets were rapidly quenched cooled from a temperature of around 1650° C. to room temperature (from 15° C. to 20° C.) for a period of 1 to 2 seconds in order to form solid particles of the hydrocarbon synthesis catalyst having a particle size of from 0.5 microns to 250 microns. The solid particles were then dried in a rotary oven at 120° C. at a feed rate of 0.5 kg/hour.

The amounts of each of the above promoters in the dried solid particles produced at 75 bar are set out in the table below:

TABLE 1
Composition of the quench cooled and dried hydrocarbon
synthesis catalyst produced at 75 bar.

| Ingredient | Amount (wt %) |
|---|---|
| CaCO₃ | 1.71 |
| MgCO₃ | 0.96 |
| Na₂CO₃ | 0.51 |
| K₂CO₃ | 0.38 |
| SiO₂ | 0.71 |
| Al₂O₃ | 0.19 |
| MnO₂ | 0.39 |
| Total Fe | 71.00 |
| Alkalinity Index ^{(note a)} | 99 |

^{(note a)}
Alkalinity Index = [wt %(Na₂CO₃ + K₂CO₃)/wt %(SiO₂ + Al₂O₃)] × 100

### Example 2

### Particle Size of a Hydrocarbon Synthesis Catalyst Prepared According to the Invention

The particle size distributions of the rapidly quenched hydrocarbon synthesis catalyst are set out in Table 2 below, the varying particle sizes being achieved by varying the pressure of the water during atomization. It is envisaged that the method according to the invention provides the advantage that one does not have to go through the steps of casting, crushing, milling, classification and cyclone separation to achieve a catalyst having a particular particle size and that this can quite economically be achieved by varying the water pressure as demonstrated in Table 2 below.

TABLE 2
Particle Size Distribution of rapidly quenched HTFT
catalyst produced at different water pressures.

Percentage smaller than

| | 50 bar | 75 bar | 100 bar | 150 bar |
|---|---|---|---|---|
| Size | water | water | water | water |
| (micron) | pressure | pressure | pressure | pressure |
| 249 | 100 | 100 | 100 | 100 |
| 176 | 94 | 100 | 99 | 100 |
| 125 | 78 | 70 | 95 | 85 |
| 88 | 56 | 48 | 85 | 71 |
| 62 | 33 | 30 | 70 | 57 |
| 44 | 14 | 19 | 55 | 46 |
| 31 | 3 | 11 | 41 | 35 |
| 22 | 1 | 6 | 29 | 25 |
| 16 | 0.5 | 3 | 19 | 18 |
| 11 | 0 | 1 | 11 | 10 |
| 7.8 | 0 | | 7 | 6 |
| 5.5 | 0 | | 4 | 3 |
| 3.9 | 0 | | 1 | 1 |
| 2.8 | 0 | | 0.4 | 0.2 |

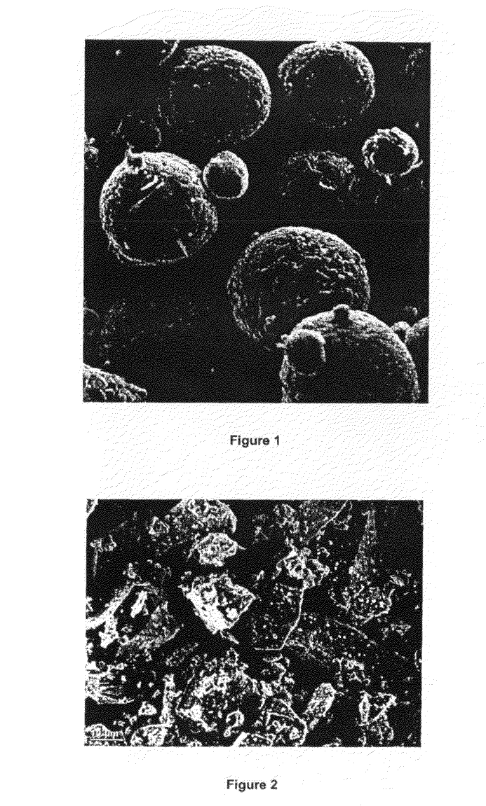

A further advantage of the method according to the invention is that the particles of the hydrocarbon synthesis catalyst are substantially spherical in nature as shown in FIG. 1 (by virtue of Scanning Electron Microscopy (SEM)) and it is envisaged that the spherical nature of the particles will improve the flow properties of the catalyst when used in an FT process.

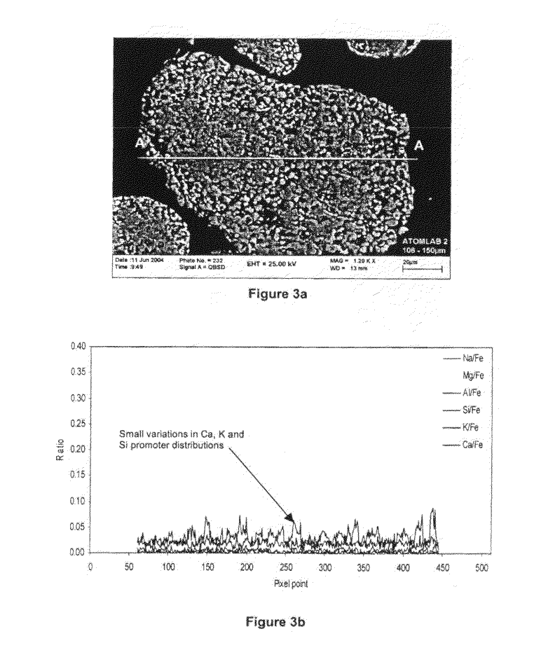

This particular advantage is well demonstrated when compared to an SEM micrograph of the standard fused catalyst of Example 1, which morphology is shown in FIG. 2. It will be clear that the shape of the particles of the conventional fused, crushed and ball milled catalyst are highly irregular.

### Example 3

### Homogenous Distribution of Catalyst Promoters in a Hydrocarbon Synthesis Catalyst Prepared According to the Invention

As discussed above, a further advantage of the method according to the invention is that the distribution of catalyst promoters, in the particles of the hydrocarbon synthesis catalyst prepared according to the invention, is substantially homogeneous thereby providing similar if not better hydrocarbon synthesis performance in an FT process. This is demonstrated below.

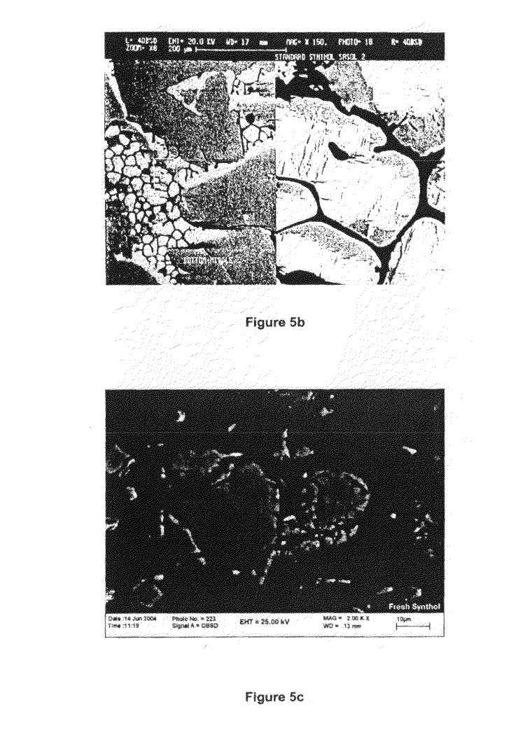

Further, the selectivity of each particle of hydrocarbon synthesis catalyst prepared according to the method of the invention will have substantially the same hydrocarbon selectivity since the promoter compositions of the catalyst particles (alkalinity indices) are substantially the same for the different particle fractions (see Table 4 below) whereas the alkalinity indices for the particle fractions of a standard fused and milled catalyst known in the art and shown in Table 3 below, differ significantly and hence will give different hydrocarbon selectivities. All promoters, including the key alkali chemical promoters are fed together with the iron oxides to the fusion furnace. As mentioned before, most iron oxides used (mill scales) are contaminated with some silica. Neither the K⁺ nor the Si⁴⁺ ions can enter into solid solution with the magnetite and so on cooling during casting in the conventional manner, small occlusions of alkali/silicates are present as separate phases in the cooled ingots (black lines in FIGS. 5a and 5b)

TABLE 3
Chemical composition of catalyst fractions for
the standard fused catalyst of Example 1.

| | Standard fused fractions | | | | |
|---|---|---|---|---|---|
| | +150 | 75-150 | 45-75 | −45 | |
| Composition | micron | micron | micron | micron | Average |
| Na₂CO₃ | 0.46 | 0.48 | 0.55 | 0.65 | 0.54 |
| K₂CO₃ | 0.34 | 0.40 | 0.53 | 0.96 | 0.56 |
| SiO₂ | 0.68 | 0.79 | 0.90 | 1.21 | 0.90 |
| Al₂O₃ | 0.25 | 0.24 | 0.25 | 0.24 | 0.25 |
| Alkalinity Index | 86 | 85 | 94 | 111 | 96 |

TABLE 4
Chemical composition of catalyst fractions for the hydrocarbon
synthesis catalyst of the invention, produced at 75 bar.

| | Atomised Catalyst Fractions | | | | |
|---|---|---|---|---|---|
| | +150 | 75-150 | 45-75 | −45 | |
| Composition | micron | micron | micron | micron | Average |
| Na₂CO₃ | 0.50 | 0.50 | 0.51 | 0.51 | 0.51 |
| K₂CO₃ | 0.37 | 0.37 | 0.38 | 0.39 | 0.38 |
| SiO₂ | 0.70 | 0.71 | 0.70 | 0.70 | 0.70 |
| Al₂O₃ | 0.20 | 0.19 | 0.19 | 0.20 | 0.20 |
| Alkalinity Index | 97 | 97 | 100 | 100 | 99 |

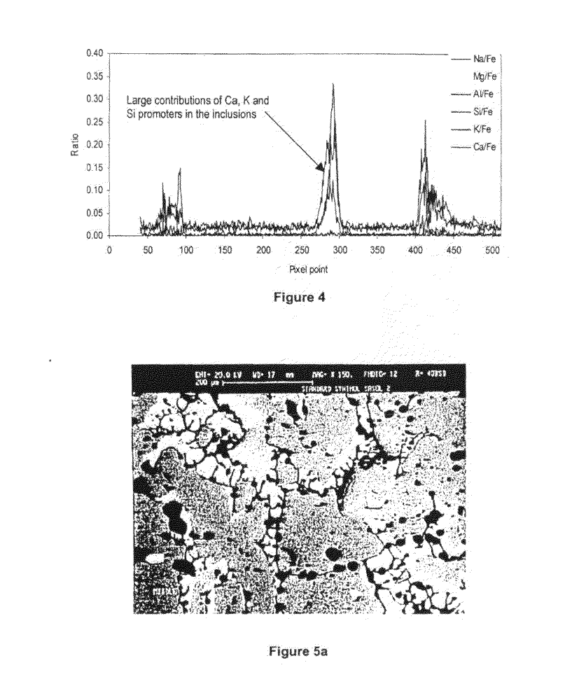

Table 4 above, as well as the Scanning Electron Micrograph and Energy Dispersive X-Ray (EDX) analysis shown in FIGS. 3a and 3b respectively demonstrate that the catalyst promoters are substantially homogeneously distributed in particles of all sizes of the quench cooled catalyst prepared according to the method of the invention. This is shown in FIG. 3a of the catalyst prepared according to the invention wherein it will be clear that the particles thereof contain a more uniform distribution of finer inclusions than those inclusions found in the standard fused catalyst of Example 1 (FIG. 5). The catalyst promoters, namely Na, Mg, Al, Si, K and Ca are all homogeneously distributed across the atomized catalyst (shown in FIG. 3b, the distribution taken though the cross section of that atomized particle shown in FIG. 3a, the cross section being shown by line A-A) compared to the standard fused catalyst where there is segregation of the Ca, Si and K promoters to the large inclusions which are characteristic of the standard fused catalyst of Example 1 and cast catalysts known in the art (shown in FIGS. 4 and 5a and 5b).

More specifically, when the catalyst of the invention is compared to the standard fused catalyst, the EDX line scans obtained through the cross-sections of a typical fused particle is given in FIG. 4 and shows evidence of the non-uniform distribution of promoters across the particle. The inclusions or grain boundaries show much higher concentrations of calcium, potassium and silicon compared to the bulk iron.

### Example 4

### Mechanical Strength of the Hydrocarbon Synthesis Catalyst Prepared According to the Invention

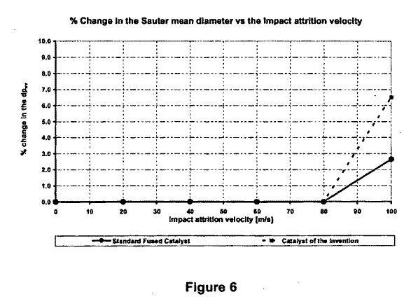

Yet a further advantage of the method according to the invention is that the hydrocarbon synthesis catalyst particles (indicated by the red line in FIG. 6) have a similar mechanical strength when compared to the standard fused catalyst of Example 1 (indicated by the solid line in FIG. 6). This is demonstrated in FIG. 6.

### Example 5

### Selectivity of the Hydrocarbon Synthesis Catalyst Prepared According to the Invention

The catalyst of the invention was prepared according to the method described in example 1, however it was then reduced in a reactor that was heated to a temperature of 380° C. under nitrogen flow. 2 kg of catalyst was loaded when the temperature reached about 330° C. The reactor pressure was maintained at 18 bar for the entire reduction period. The reduction sequence was started by cutting in the hydrogen to displace the nitrogen, while maintaining a certain linear velocity. As usual, the reduction period was set for 16 hours, while water draining was done every hour to monitor the rate of reduction.

The hydrocarbon synthesis catalyst prepared as described above was tested in a 50 mm Pilot Plant fixed fluidised HTFT reactor. The synthesis reaction experiments were executed at 350° C. and 25 bar and gave stable performance between day 2 and 5 for comparison with the standard fused baseline catalysts which were tested at the same conditions.

TABLE 5
Reactor conditions and run results for the hydrocarbon synthesis
catalyst of the invention vs. the standard fused catalyst

| | | Catalyst | standard |
|---|---|---|---|
| | | of the | fused |
| | | invention | catalyst |
| | | A2515 | Typical Range |
| Period (days) | | P2-5 | P2-5 |
| Temperature (° C.) | | 350.00 | 350.00 |
| Pressure (bar) | | 25.00 | 25.00 |
| Conversion (overall): | CO + CO2 | 88.2 | 80-86 |
| Per pass conversions: | CO + CO2 | 58.0 | 38-50 |
| Partial pressures (bara): Inlet | H2 | 13.1 | 11.5-12.7 |
| | CO | 3.7 | 2.5-3.4 |
| | CO2 | 1.3 | 1.0-2.5 |
| | H2/CO Ratio | 3.5 | 3.3-4.5 |
| Outlet | H2 | 9.1 | 8.2-9.8 |
| | CO | 0.3 | 0.3-0.7 |
| | CO2 | 2.0 | 1.8-3.3 |
| | H2O | 2.9 | 2.5-3.0 |
| Mohl/hr CO + CO2 converted (overall) | | 130 | 105-130 |
| Oil Acid number (mg KOH/g oil) | | 8.9 | 5.5-12.5 |
| Water Acid number (mg KOH/g water) | | 7.4 | 8.2-13.0 |
| Olef/Par: C2 ratio | | 1.3 | 1.2-1.8 |
| Olef/Par: C3 ratio | | 6.2 | 6.0-8.0 |
| Carbon atom selectivity (%) | CH4 | 10.7 | 5.0-12.0 |
| | C2H4 | 4.1 | 3.3-5.4 |
| | C2H6 | 3.1 | 2.3-3.4 |
| | C3H6 | 8.7 | 7.0-10.0 |
| | C3H8 | 1.4 | 1.0-1.5 |
| | C5+ | 60.4 | 50-60 |

### Example 6

A consideration of carbon formation results for the end of run standard fused catalysts and catalysts of the invention after synthesis tests is given in Table 6 below and will show yet a further advantage of the method according to the invention, namely that by using a catalyst prepared according to the invention, less elemental carbon is produced when compared to that of a standard fused catalyst in an HTFT process. The catalysts indicated in Table 6 were all tested (average of 5 synthesis tests) at the same synthesis conditions shown in example 5 above.

TABLE 6
Summary on the comparison of the average Elemental Carbon
Formation Rate for standard fused catalysts and hydrocarbon
synthesis catalysts of the invention.

| | Average Carbon Formation Rate | | |
|---|---|---|---|
| | for 5 synthesis tests | | |
| | Standard | Hydrocarbon | Delta |
| | fused | synthesis catalyst | Average |
| | catalyst | of the invention | % |
| g Elemental Carbon/100 g | 24 | 18 | −25 |
| Fe/day | | | |

The carbon formation rate during the HTFT synthesis reaction for the catalysts of the invention results in 25% less elemental carbon compared to that of the standard fused catalyst. This means that the lifetime of the catalyst of the invention is potentially 25% longer than a standard fused catalyst and hence a saving of 25% on the amount of fresh catalyst consumed.

At FT operating temperatures below 240° C. (Typical LTFT conditions), little elemental carbon is deposited on the catalyst. During HTFT operation (about 280 to 350° C.) with iron based catalysts, however, elemental carbon, as distinct from carbonaceous coke, is deposited throughout the synthesis run at a fairly constant rate. The two source reactions could be:

2CO→C+CO₂ (The Boudouard reaction)

or CO+H₂→C+H₂O

The boudouard reaction is considered to be the key reaction resulting in elemental carbon deposition because of its lower Gibbs free energy and the fact that the rate of carbon deposition is markedly depressed by higher hydrogen partial pressures. When elemental carbon forms in iron catalysts, the density of the particles is lowered and because of the vigorous movement of the catalyst particles in the high gas velocity fluidised beds, catalyst particles break up and carbon and potassium rich fines are produced. As shown in example 3 above, the finer fractions of the standard catalyst contain high amounts of potassium and silica. The loss of these fines hence means that alkali is lost from the reactors which then contribute to a decline in activity of the SAS reactors. Fresh catalyst has to be added more frequently to sustain activity.

Another disadvantage of high elemental carbon make on the standard fused catalyst is that it will change the powder fluidization characteristics in fluidised bed operation. The commercial Sasol dense phase turbulent fluidised bed reactors, called the Sasol advanced Synthol (SAS reactors) utilize a reduced and promoted iron oxide Geldart group A powder catalyst, i.e. the standard fused and milled catalyst. It is possible for a group A powder to change to either a group B or C powder depending on the process conditions and the extent to which the particle properties change in situ. A group A powder can potentially change to a group B powder as a result of the loss of fines, hence resulting in a coarser particle size distribution. Because group B powder de-aerates very rapidly, the flow regime in the dipleg of the cyclones can quickly change from dense phase flow to eventual de-fluidization. This would cause a reduction in the pressure recovery and can lead to eventual de-fluidization of the catalyst in the dipleg, causing the dipleg to block and eventually the reactor has to be shut down. Similarly, it is also possible for a powder having a group A classification to change to one having a group C classification (e.g. through the accumulation of fines). This would result in a lower flow coefficient and hence a higher catalyst level will be required in the dipleg to ensure that the necessary pressure recovery is established. The residence time of the catalyst in the dipleg is increased and hence the risk of blocking the dipleg is also increased. The increase in fines fraction as a result of catalyst break-up due to carbon lay down causes an increase in the bed voidage and if allowed to continue uncontrolled can result in a fluidization regime transition from the turbulent regime to the transport regime (fast fluidization). This must be avoided because the uncontrolled bed expansion and transport of catalyst causes the cyclones to block followed by a reactor shut down. An on-line catalyst removal (used catalyst) and catalyst addition (fresh) policy was implemented to maintain the fluidised bed densities of the SAS reactors within certain limits to prevent the above occurrences. Hence, if the elemental carbon formation rate is 25% lower than that of the standard fused catalyst, as indicated by the catalyst prepared according to the invention, 25% less fresh catalyst has to be added to maintain fluidised bed densities, activity and hydrocarbon selectivity.

It will be appreciated that many variations in detail are possible without thereby departing from the scope and spirit of the invention.

1. A method for the preparation of a hydrocarbon synthesis catalyst, the method including the steps of:
(a) providing a melt including a mixture of at least one metal oxide and a catalyst promoter selected from the group consisting of at least one of a source of an alkali metal and a source of an alkali earth metal;
(b) subjecting the melt to a fluid stream so as to thereby disperse the melt into droplets including the metal oxide and the catalyst promoter; and
(c) quenching the droplets of the melt so as to form the hydrocarbon synthesis catalyst in the form of solid particles including the metal oxide and the catalyst promoter.

2. The method of claim 1, wherein the metal oxide is magnetite (Fe₃O₄).

3. The method of claim 1, wherein the metal oxide may comprise a mixture of iron oxides.

4. The method of claim 1, wherein the hydrocarbon synthesis catalyst contains between 68% to 73% of total iron metal.

5. The method of claim 1, wherein the source of alkali metal is selected from a source of elements of Group IA and the alkali earth metal is selected from a source of elements of Group IIA.

6. The method of claim 1, wherein the catalyst promoter comprises a mixture of a source of alkali metals and a source of earth alkali metals.

7. The method of claim 1, wherein the fluid stream comprises gas or liquid.

8. The method of claim 1, wherein the fluid stream is water at a pressure of 50 to 150 bar.

9. The method of claim 1, wherein an atomizer is used to disperse the melt into droplets.

10. The method of claim 1, wherein the droplets of the melt are cooled from a temperature of from 1600° C. to 1700° C. to a temperature of 15° C. to 20° C.

11. The method of claim 1, wherein the solid particles are substantially spherical in shape.

12. The method of claim 1, wherein the solid particles of the hydrocarbon synthesis catalyst of step (c) have a particle size range of 0.5 to 500 micron.

13. The method of claim 1, wherein the solid particles of the hydrocarbon synthesis catalyst of step (c) have a BET surface area of from 1 m²/g to 5 m²/g.

14. The method of claim 1, wherein the catalyst promoter is homogeneously distributed within the solid particles.

15. The method of claim 1, wherein the hydrocarbon synthesis catalyst of step (c) is subjected to a heat treatment step wherein the metal oxide is reduced to metal in the zero oxidation state to form a reduced hydrocarbon synthesis catalyst in the form of solid particles.

16. The method of claim 20, wherein the solid particles of the reduced hydrocarbon synthesis catalyst have a BET surface area of from 20 m²/g to 30 m²/g.

17. A two phase High Temperature Fischer Tropsch process for the conversion of a feed of H₂ and at least one carbon oxide to hydrocarbons containing at least 40% on a mass basis of hydrocarbons with five or more carbon atoms; the conversion being carried out by contacting the H₂ and the at least one carbon oxide in the presence of a hydrocarbon synthesis catalyst prepared by a method comprising the steps of:
(a) providing a melt including a mixture of at least one metal oxide and a catalyst promoter selected from the group consisting of at least one of a source of an alkali metal and a source of an alkali earth metal;
(b) subjecting the melt to a fluid stream so as to thereby disperse the melt into droplets including the metal oxide and the catalyst promoter;
(c) quenching the droplets of the melt so as to form the hydrocarbon synthesis catalyst in the form of solid particles including the metal oxide and the catalyst promoter; and
(d) subjecting the solid particles of the hydrocarbon synthesis catalyst of step (c) to a heat treatment step so as reduce the metal oxide to a metal having an oxidation state of zero.
